(19) United States
(12) Patent Application Publication
(10) Pub. No.: US 2026/0232613 A1
(43) Pub. Date: Aug. 13, 2026

(54) Dietary Supplement Formulation for Alcohol Metabolism, Hangover Prevention, Stress Recovery, and Immune System Support

(71) Applicant: Good Mana LLC, Honolulu, HI (US)

(72) Inventor: Denny KWOCK, Honolulu, HI (US)

(73) Assignee: Good Mana LLC, Honolulu, HI (US)

(21) Appl. No.: 19/535,051

(22) Filed: Feb. 10, 2026

Related U.S. Application Data
(60) Provisional application No. 63/834,073, filed on Feb. 12, 2025.

(51) Int. Cl. A61K 31/198; A61K 9/00; A61K 31/375; A61K 45/06

(52) U.S. Cl. CPC A61K 31/198; A61K 9/0053; A61K 31/375; A61K 45/06

(57) ABSTRACT

A dietary supplement composition contains at least one of the group consisting of turmeric powder, curcumin extract, black pepper fruit extract, N-acetylcysteine, and Vitamin C as active ingredients. The active ingredients act synergistically to promote efficient ethanol metabolism, mitigate hangovers from alcohol consumption, enhance immune function, and/or promote stress recovery.

### CROSS-REFERENCING

This application claims the benefit of provisional application No. 63/834,073, which was filed on Feb. 12, 2025, which is all hereby incorporated in reference by its entirety for all purposes.

### FIELD

Among other things, the present invention is related to the field of dietary supplements. More specifically, the present invention is related to oral formulations that include a combination of dietary supplements for human consumption to achieve a number of benefits: support alcohol metabolism, reduce physiological stress responses, and promote immune system.

### BACKGROUND

Alcohol consumption results in the formation of metabolic byproducts, some of which are harmful. One notable example is acetaldehyde that can contribute to hangover symptoms and oxidative stress. On the other hand, both alcohol consumption and physical or mental exertion deplete critical antioxidants and induce inflammatory responses that can impair recovery. Alcohol intake and stress can also suppress immune function, making individuals more susceptible to infections. While individual nutrients have been studied for their potential to mitigate these effects, a formulation that combines specific bioactive compounds to enhance their collective efficacy has not been sufficiently explored.

The present invention provides, among many other, a solution to tackle these problems. Specifically, the present invention provides a dietary supplement formulation designed to improve alcohol metabolism, reduce hangover severity, enhance immune function, and promote overall physiological resilience.

### SUMMARY OF INVENTION

The following brief summary is not intended to include all features and aspects of the present invention. The present invention is related to dietary supplements and their applications.

One aspect of the invention is the means that have beneficial effects to the aftermath from alcohol consumption, specifically, a dietary supplement composition that comprises at least one of the group consisting of turmeric powder, curcumin extract, black pepper fruit extract, N-acetylcysteine, and Vitamin C as active ingredients. The active ingredients act synergistically to promote efficient ethanol metabolism, mitigate hangovers from alcohol consumption, enhance immune function, and/or promote stress recovery.

In the dietary supplement composition of the invention, the active ingredients can contain the turmeric powder in an amount between 200 mg and 2000 mg.

The active ingredients can also contain the curcumin extract in an amount between 10 mg and 300 mg, the black pepper fruit extract in an amount between 5 mg and 50 mg, the N-acetylcysteine in an amount between 100 mg and 1200 mg, and/or the Vitamin C in an amount between 0 mg and 1000 mg.

With the dietary supplement composition of the invention, the black pepper fruit extract provides piperine that can enhance the bioavailability of curcumin by at least 10 folds. Further, the N-acetylcysteine and Vitamin C can act synergistically to enhance antioxidant protection against alcohol-induced oxidative stress and boost immune function.

In one embodiment, the dietary supplement composition can comprise 885 mg turmeric powder, 100 mg curcumin extract, 15 mg black pepper fruit extract, 500 mg N-acetylcysteine, and 100 mg Vitamin C.

In another embodiment, the dietary supplement composition can comprise 885 mg turmeric powder, 100 mg curcumin extract, 15 mg black pepper fruit extract, and 500 mg N-acetylcysteine.

In yet another embodiment, the dietary supplement composition can comprise 885 mg turmeric powder, 500 mg N-acetylcysteine, and 100 mg Vitamin C.

In still another embodiment, the dietary supplement composition can comprise 885 mg turmeric powder and 500 mg N-acetylcysteine.

Another aspect of the invention is related to a method of promoting efficient ethanol metabolism, mitigating hangovers from alcohol consumption, enhancing immune function, and/or promoting stress recovery. The method comprises administering orally a dietary supplement composition to a subject in need thereof. Further, the dietary supplement composition can include turmeric powder, curcumin extract, black pepper fruit extract, N-acetylcysteine, Vitamin C, or a combination thereof.

With the just-described method, the turmeric powder can be in an amount between 200 mg and 2000 mg, the curcumin extract can be in an amount between 10 mg and 300 mg, the black pepper fruit extract can be in an amount between 5 mg and 50 mg, the N-acetylcysteine can be in an amount between 100 mg and 1200 mg, and the Vitamin C can be in an amount between 0 mg and 1000 mg.

Further with the method, the above-described embodiments of the dietary supplement composition can be used for oral administration. Likewise, the black pepper fruit extract provides piperine that can enhance the bioavailability of curcumin by at least 10 folds; and the N-acetylcysteine and Vitamin C act synergistically to enhance antioxidant protection against alcohol-induced oxidative stress and boost immune function.

The details of the invention are set forth in the description below. Other features, objects, and advantages of the invention will be apparent to those persons skilled in the art upon reading the the description, as well as from the appended claims.

### DETAILED DESCRIPTION

The following detailed description illustrates some embodiments of the invention by way of example and not by way of limitation. The section headings and any subtitles used herein are for organizational purposes only and are not to be construed as limiting the subject matter described in any way. The contents under a section heading and/or subtitle are not limited to the section heading and/or subtitle, but apply to the entire description of the present invention.

### Definitions

The following definitions are set forth to illustrate and describe the meaning and scope of (a) certain embodiments of the invention and (b) certain terms used in the section of Detailed Description.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The term “oral compositions” or “oral formulations” as used herein refers to formulations deliverable to a mammal via the oral cavity, mouth, throat, nasal passages, or any combination thereof. Nonlimiting examples of these compositions include liquids or solid dosage forms. The former includes beverages, supplemental water, liquid or gel formulations (specifically those deliverable directly to the mouth and throat), foams, and saline washes. The latter covers pills, soft gels, tablets, capsules, chews, films, and center-filled formats (gums, lozenges, or compressed tablets). These preparations may be administered using various delivery devices and packaging, including but not limited to: droppers, liquid pumps, cups, and bottles, sprayers (including pressurized versions), atomizers, and air inhalation devices. Additionally, sprayers, atomizers, and inhalers may be integrated with a battery or electric power source.

The term “preservation” or “preserve” as used herein refers to the process or action that prevents or slows the decomposition or undesirable chemical change of at least one component of a sample.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function may additionally or alternatively be described as being operative to perform that function.

As used herein, the phrase, “for example,” the phrase, “as an example,” and/or simply the terms “example” and “exemplary” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, the phrases “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) may refer to A alone, B alone, or the combination of A and B.

As used herein, the term “and/or” placed between a first entity and a second entity means one of (1) the first entity, (2) the second entity, and (3) the first entity and the second entity.

As described herein, a dietary supplement of the invention can contain turmeric powder. Turmeric is a bright yellow-orange spice derived from the rhizome (underground stem) of Curcuma longa, a plant in the ginger family. It has been used for centuries in cooking and traditional medicine, especially in South and Southeast Asia. Turmeric powder can be made directly using turmeric plant harvested from Hawaii farms and dried into powder without using much heat. In some cases, turmeric powder can be obtained commercially.

In some cases, a dietary supplement of the invention can contain any appropriate amount of turmeric powder. For example, at least 5 percent (e.g., at least 10, 15, 20, 25, 30, 35, 40, 50, 60, 70, 80, or 90 percent) of the dietary supplement can be the turmeric powder. Typically, the dietary supplement contains between 100 mg and 5000 mg (e.g., between 100 mg and 3000 mg, between 100 mg and 2000 mg, between 100 mg and 1000 mg, between 100 mg and 500 mg, between 200 mg and 3000 mg, between 200 mg and 2000 mg, between 200 mg and 1500 mg, between 200 mg and 1000 mg, between 500 mg and 2000 mg, and between 500 mg and 1000 mg) of the turmeric powder. In some cases, the dietary supplement can be formulated to contain an amount of the turmeric powder such that a daily dose of between 100 mg and 5000 mg (e.g., between 100 mg and 3000 mg, between 100 mg and 2000 mg, between 100 mg and 1000 mg, between 100 mg and 500 mg, between 200 mg and 3000 mg, between 200 mg and 2000 mg, between 200 mg and 1500 mg, between 200 mg and 1000 mg, between 500 mg and 2000 mg, and between 500 mg and 1000 mg) of the turmeric powder can be conveniently administered.

As described herein, a dietary supplement of the invention can contain curcumin extract. Curcumin belongs to a class of compounds called curcuminoids found in turmeric. Curcuminoids mainly include curcumin, demethoxycurcumin (DMC), and disdemethoxycurcumin (BDMC)). Curcumin is a diarylheptanoid, being either in enol form or keto form:

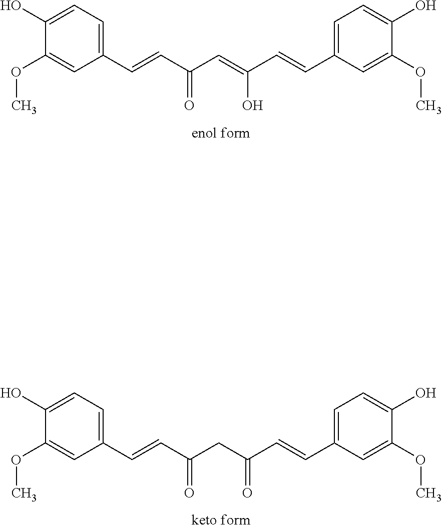 [SMILES: COc1cc(/C=C/C(=O)/C=C(O)/C=C/c2ccc(O)c(OC)c2)ccc1O ; COc1cc(/C=C/C(=O)CC(=O)/C=C/c2ccc(O)c(OC)c2)ccc1O]

Curcuminoids can be synthesized or derived from natural sources such as turmeric. About 70-80% of curcuminoids is curcumin. Curcumin extract is a concentrated preparation obtained from turmeric. Compared to the turmeric powder, curcumin extract contains curcumin at much higher and standardized levels. Curcumin extract can be obtained commercially.

In some cases, the dietary supplement can contain any appropriate amount of curcumin extract, such as a standard turmeric extract. For example, at least 5 percent (e.g., at least 10, 15, 20, 25, 30, 35, 40, 50, 60, 70, 80, or 90 percent) of the dietary supplement can be the curcumin extract. Typically, the dietary supplement contains between 10 mg and 500 mg (e.g., between 25 mg and 500 mg, between 50 mg and 500 mg, between 100 mg and 500 mg, between 10 mg and 100 mg, between 10 mg and 200 mg, between 10 mg and 300 mg, between 50 mg and 200 mg, between 50 mg and 300 mg, between 100 mg and 200 mg, and between 100 mg and 300 mg) of the curcumin extract. In some cases, the dietary supplement can be formulated to contain an amount of the curcumin extract such that a daily dose of between 10 mg and 500 mg (e.g., between 25 mg and 500 mg, between 50 mg and 500 mg, between 100 mg and 500 mg, between 10 mg and 100 mg, between 10 mg and 200 mg, between 10 mg and 300 mg, between 50 mg and 200 mg, between 50 mg and 300 mg, between 100 mg and 200 mg, and between 100 mg and 300 mg) of the curcumin extract can be conveniently administered.

As described herein, a dietary supplement of the invention can contain black pepper fruit extract. Black pepper fruit extract is a concentrated preparation obtained from the dried fruits (peppercorns) of Piper nigrum. It is typically standardized to contain piperine, the primary bioactive alkaloid responsible for black pepper's pungency. Black pepper fruit extract can be obtained commercially.

In some cases, the dietary supplement can contain any appropriate amount of black pepper fruit extract. For example, at least 1 percent (e.g., at least 2, 5, 10, 15, 20, 25, 30, 35, 40, 50, 60, 70, 80, or 90 percent) of the dietary supplement can be the black pepper fruit extract. Typically, the dietary supplement contains between 1 mg and 100 mg (e.g., between 5 mg and 10 mg, between 5 mg and 20 mg, between 5 mg and 50 mg, between 5 mg and 75 mg, between 5 mg and 100 mg, between 10 mg and 20 mg, between 10 mg and 50 mg, between 10 mg and 100 mg, between 20 mg and 50 mg, and between 20 mg and 100 mg) of the black pepper fruit extract. In some cases, the dietary supplement can be formulated to contain an amount of the black pepper fruit extract such that a daily dose of between 1 mg and 100 mg (e.g., between 5 mg and 10 mg, between 5 mg and 20 mg, between 5 mg and 50 mg, between 5 mg and 75 mg, between 5 mg and 100 mg, between 10 mg and 20 mg, between 10 mg and 50 mg, between 10 mg and 100 mg, between 20 mg and 50 mg, and between 20 mg and 100 mg) of the black pepper fruit extract can be conveniently administered.

As described herein, a dietary supplement of the invention can acetylcysteine or N-acetylcysteine (NAC). N-acetylcysteine is a chemically modified form of the amino acid cysteine in which an acetyl group is attached to the nitrogen atom.

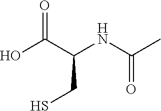

It serves as a precursor to glutathione, one of the body's key antioxidants, and is widely used in medicine and research for its mucolytic (mucus-thinning) and antioxidant properties. N-acetylcysteine can be obtained commercially.

In some cases, the dietary supplement can contain any appropriate amount of N-acetylcysteine. For example, at least 5 percent (e.g., at least 10, 15, 20, 25, 30, 35, 40, 50, 60, 70, 80, or 90 percent) of the dietary supplement can be the N-acetylcysteine. Typically, the dietary supplement contains between 10 mg and 1500 mg (e.g., between 50 mg and 500 mg, between 50 mg and 1000 mg, between 50 mg and 1500 mg, between 100 mg and 500 mg, between 100 mg and 1000 mg, between 100 mg and 1200 mg, between 100 mg and 1500 mg, between 200 mg and 500 mg, between 200 mg and 1200 mg, and between 200 mg and 1500 mg) of the N-acetylcysteine. In some cases, the dietary supplement can be formulated to contain an amount of the N-acetylcysteine such that a daily dose of between 10 mg and 1500 mg (e.g., between 50 mg and 500 mg, between 50 mg and 1000 mg, between 50 mg and 1500 mg, between 100 mg and 500 mg, between 100 mg and 1000 mg, between 100 mg and 1200 mg, between 100 mg and 1500 mg, between 200 mg and 500 mg, between 200 mg and 1200 mg, and between 200 mg and 1500 mg) of the N-acetylcysteine can be conveniently administered.

As described herein, a dietary supplement of the invention can contain Vitamin C (ascorbic acid). It is used as a food additive and a dietary supplement for its antioxidant properties.

In some cases, the dietary supplement can contain any appropriate amount of Vitamin C. For example, optionally at least 1 percent (e.g., at least 2, 5, 10, 15, 20, 25, 30, 35, 40, 50, 60, 70, 80, or 90 percent) of the dietary supplement can be the Vitamin C. Typically, the dietary supplement contains between 0 mg and 2000 mg (e.g., between 0 mg and 1000 mg, between 10 mg and 500 mg, between 10 mg and 1000 mg, between 25 mg and 500 mg, between 25 mg and 1000 mg, between 50 mg and 1000 mg, between 50 mg and 2000 mg, between 100 mg and 500 mg, between 100 mg and 1000 mg, and between 100 mg and 2000 mg) of the Vitamin C. In some cases, the dietary supplement can be formulated to contain an amount of the Vitamin C such that a daily dose of between 0 mg and 2000 mg (e.g., between 0 mg and 1000 mg, between 10 mg and 500 mg, between 10 mg and 1000 mg, between 25 mg and 500 mg, between 25 mg and 1000 mg, between 50 mg and 1000 mg, between 50 mg and 2000 mg, between 100 mg and 500 mg, between 100 mg and 1000 mg, and between 100 mg and 2000 mg) of the Vitamin C can be conveniently administered.

### Dietary Supplement Embodiments

One aspect of the present invention provides a collection of dietary supplement embodiments.

A dietary supplement of the present invention can be formulated to have a particular dose. For example, the dietary supplement can be in the form of a capsule or tablet with a total weight that is between about 150 mg and about 2000 mg (e.g., between about 200 mg and about 800 mg, between about 250 mg and about 1000 mg, between about 250 mg and about 1500 mg, between about 500 mg and about 1000 mg, between about 500 mg and about 1500 mg, or between about 500 mg and about 2000 mg).

As described herein, a single dietary supplement of the present invention (e.g., a capsule or tablet) can be formulated in a manner that helps maintain the stability of the component within the composition. In some cases, a single dietary supplement of the present invention can be coated with at coating or placed into a capsule having the ability to deliver the contents to the intestines of a mammal following oral administration. For example, the dietary supplement can be designed to release its contents when the composition reaches a location of the intestines having a pH above about 6.8.

In some embodiment, the dietary supplement can include about 885 mg turmeric powder, about 100 mg curcumin extract, 15 mg black pepper fruit extract, 500 mg N-acetylcysteine, 100 mg Vitamin C.

In another embodiments, the dietary supplement can include about 885 mg turmeric powder, about 100 mg curcumin extract, about 15 mg black pepper fruit extract, about 500 mg N-acetylcysteine.

In still another embodiments, the dietary supplement can include about 885 mg turmeric powder, 500 mg N-acetylcysteine, 100 mg Vitamin C.

In yet another embodiments, the dietary supplement can include about 885 mg turmeric powder, 500 mg N-acetylcysteine.

Additionally, other ingredients may be incorporated in the dietary supplement, which include, without limitation, B-vitamins, electrolytes, minerals, amino acids, or botanical extracts.

As described herein, a daily dose may be prepared and administered in the form of one or more capsules or tablets (e.g., two capsules or tablets, three capsules or tablets, and four capsules or tablets). In some cases, one or more capsules or tablets can be administered in one or more dosages over the course of 24 hours (e.g., one dose, two doses, three doses, and four doses).

The oral dietary supplement composition is formulated for administration prior to, or substantially concurrent with, the ingestion of ethanol. In some cases, a single pre-ingestion dosage is sufficient to achieve the desired metabolic support in a majority of subjects. In other cases, a multi-stage dosing regimen can be prescribed wherein a supplemental dosage is administered post-ingestion-specifically prior to a period of rest or sleep. This secondary administration may provide an augmented or synergistic effect, particularly in instances of elevated ethanol consumption where increased metabolic demand is present.

### Mechanism of Action of the Dietary Supplements

A dietary supplement of the present invention can provide broad spectrum anti-aging and anti-oxidant protection at the cellular level that may block or slow damaging of DNA, lipid, and protein molecules. In some cases, a dietary supplement of the present invention can protect many classes of bio-molecules involved in the aging process from damage.

In general, the dietary supplement, as described herein, can include, without limitation, fat-soluble antioxidants, phenolic compounds, and/or water soluble antioxidants. For example, the dietary supplement can include at least one fat-soluble antioxidant, at least one phenolic compound, and at least one water soluble antioxidant.

Specifically, the turmeric powder and curcumin extract as described in the dietary supplement are both bioactive. Particularly, curcumin, the active compound in turmeric, enhances liver detoxification by modulating alcohol dehydrogenase (ADH) and aldehyde dehydrogenase (ALDH) activity. Both the turmeric powder and curcumin extract possess strong antioxidant and anti-inflammatory properties, reducing oxidative damage from alcohol metabolism and stress, while also playing a role in immune regulation by modulating inflammatory cytokines.

The black pepper fruit extract (piperine) enhances curcumin bioavailability by inhibiting its metabolic breakdown, increasing systemic curcumin levels by up to 2000%. Additionally, it modulates detoxifying enzymes and may reduce oxidative liver injury caused by alcohol.

On the other hand, N-acetylcysteine is a precursor to glutathione, the body's primary antioxidant, which is depleted during alcohol metabolism. By replenishing glutathione stores, N-acetylcysteine enhances acetaldehyde detoxification, reduces oxidative stress-induced tissue damage, and supports immune function by promoting white blood cell activity.

Finally, Vitamin C functions as a potent antioxidant, scavenging free radicals generated by alcohol metabolism and stress. It also aids in immune recovery, enhances white blood cell function, and supports collagen synthesis, enhancing tissue repair and overall immune resilience.

### Synergistic Effects of the Dietary Supplements

The components of the dietary supplement composition can exhibit a synergistic effect when the combined efficacy in supporting alcohol metabolism is greater than the sum of the individual effects of each component. In other words, the combination of one component and the other results in an unexpectedly high degree of efficacy that exceeds additive expectations.

In one case, piperine provided by black pepper fruit extract can significantly increase the absorption and efficacy of curcumin, ensuring enhanced bioavailability and sustained anti-inflammatory effects.

In some case, N-acetylcysteine and Vitamin C together optimize antioxidant defenses, preventing oxidative damage in multiple cellular compartments while strengthening immune responses.

In other case, the combination of curcumin, N-acetylcysteine, and Vitamin C supports multi-pathway detoxification, reducing toxic acetaldehyde buildup while alleviating inflammatory responses and enhancing immune function.

The dietary supplement of the present invention is intended for a diverse population of human subjects who engage in the consumption of ethanol. The intended user base encompasses any adult subject seeking to mitigate the physiological sequelae associated with alcohol ingestion. This includes, but is not limited to, individuals participating in social, professional, or recreational activities where ethanol is consumed, such as professional networking events, social gatherings, festivals, celebratory events, and athletic-related social functions. In addition, the dietary supplement can be used by non-alcohol users, stress-recovery purpose.

### BEST MODE FOR CARRYING OUT INVENTION

The following example explains the present invention more concretely, but does not limit the range of the present invention.

To date, several open-label tests were performed and the outcomes of subjects that received the active formulation prior to sessions of excessive alcohol consumption were recorded.

### Example 1

In one study, for which data was collected from December 2024 through February 2025, five (5) different subjects were given four (4) variations of the invention formula as shown below and asked to take one of the formulas during evenings before heavy alcohol drinking, six (6) or more drinks. Subjects were also asked to do one evening without taking any formula to serve as a baseline.

Notable findings included addition of NAC to the formula contributed significantly to efficacy; and an unexpected finding was that too much NAC (1000 mg), reduced the feeling of inebriation during drinking which most subjects found undesirable.

| | | FEELING OF INEBRIATION | HANGOVER NEXT DAY |
|---|---|---|---|
| SUBJECT | FORMULA | (1-10, 10 being as usual) | (1-10, 10 being worst) |
| JI | 2 O, 1000 mg NAC, 100 mg vitamin C | 6 | 1 |
| JI | 2 O, 600 mg NAC, 100 mg vitamin C | 10 | 2 |
| JI | 2 O, 600 mg NAC, 100 mg | 10 | 1 |
| JI | 2 O, 100 mg vitamin C | 10 | 8 |
| JI | Take nothing | 10 | 9 |
| NC | 2 O, 1000 mg NAC, 100 mg vitamin C | 7 | 1 |
| NC | 2 O, 600 mg NAC, 100 mg vitamin C | 10 | 1 |
| NC | 2 O, 600 mg NAC, 100 mg | 10 | 1 |
| NC | 2 O, 100 mg vitamin C | 10 | 6 |
| NC | Take nothing | 10 | 10 |
| TT | 2 O, 1000 mg NAC, 100 mg vitamin C | 8 | 1 |
| TT | 2 O, 600 mg NAC, 100 mg vitamin C | 9 | 2 |
| TT | 2 O, 600 mg NAC, 100 mg | 10 | 2 |
| TT | 2 O, 100 mg vitamin C | 10 | 8 |
| TT | Take nothing | 10 | 9 |
| JIF | 2 O, 1000 mg NAC, 100 mg vitamin C | 8 | 1 |
| JIF | 2 O, 600 mg NAC, 100 mg vitamin C | 9 | 2 |
| JIF | 2 O, 600 mg NAC, 100 mg | 9 | 2 |
| JIF | 2 O, 100 mg vitamin C | 10 | 10 |
| JIF | Take nothing | 10 | 10 |
| DK | 2 O, 1000 mg NAC, 100 mg vitamin C | 6 | 1 |
| DK | 2 O, 600 mg NAC, 100 mg vitamin C | 10 | 1 |
| DK | 2 O, 600 mg NAC, 100 mg | 10 | 1 |
| DK | 2 O, 100 mg vitamin C | 10 | 9 |
| DK | Take nothing | 10 | 9 |

O = capsule of 885 mg turmeric powder, 100 mg curcumin extract, 15 mg black pepper fruit extract

### Example 2

In one social gathering, 8 individuals took the formulation described in Embodiment 1 prior to an evening of excessive alcohol consumption from 7 μm to 2 am. Upon waking the next morning at approximately 8 am, none of the subjects reported any hangover symptoms.

### Example 3

In another situation, 6 men participated in a one-week ski trip in Japan. For 5 successive days, they were on the slopes for 6-8 hours each day followed by dinners that involved heavy drinking, at least 7-8 drinks per person. Prior to drinking every evening, each individual took the formula described in Embodiment 2. In spite of the successive days of heavy drinking and strenuous winter sports that left some dehydrated each day, none reported hangovers or muscle aches.

### Example 4

During an overnight camping trip with 8 individuals, all took the formula of Embodiment 2 before a night of heavy drinking with each person drinking more than ½ bottle of whiskey or scotch each. Most drank until they blacked out. In the morning, none reported any hangover symptoms.

In some embodiments, the data (e.g. images of the sample) related to the sample is sent to the cloud without processing by the mobile device and further analysis can be conducted remotely. In some embodiments, the data related to the sample is processed by the mobile device and the results are sent to the cloud. In some embodiments, both the raw data and the results are transmitted to the Cloud.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

It is believed that the following claims particularly point out certain combinations that are directed to one of the disclosed inventions and are novel and nonobvious. Inventions embodied in other combinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A dietary supplement composition, comprising at least one of the group consisting of turmeric powder, curcumin extract, black pepper fruit extract, N-acetylcysteine, and Vitamin C as active ingredients, wherein the active ingredients act synergistically to promote efficient ethanol metabolism, mitigate hangovers from alcohol consumption, enhance immune function, and/or promote stress recovery.

2. The dietary supplement composition of claim 1, wherein the active ingredients comprise the turmeric powder in an amount between 200 mg and 2000 mg.

3. The dietary supplement composition of claim 1, wherein the active ingredients comprise the curcumin extract in an amount between 10 mg and 300 mg.

4. The dietary supplement composition of claim 1, wherein the active ingredients comprise the black pepper fruit extract in an amount between 5 mg and 50 mg.

5. The dietary supplement composition of claim 1, wherein the active ingredients comprise the N-acetylcysteine in an amount between 100 mg and 1200 mg.

6. The dietary supplement composition of claim 1, wherein the active ingredients comprise the Vitamin C in an amount between 0 mg and 1000 mg.

7. The dietary supplement composition of claim 1, wherein the black pepper fruit extract provides piperine that enhances the bioavailability of curcumin by at least 10 folds.

8. The dietary supplement composition of claim 1, wherein the N-acetylcysteine and Vitamin C act synergistically to enhance antioxidant protection against alcohol-induced oxidative stress and boost immune function.

9. The dietary supplement composition of claim 1, wherein the active ingredients comprise 885 mg turmeric powder, 100 mg curcumin extract, 15 mg black pepper fruit extract, 500 mg N-acetylcysteine, and 100 mg Vitamin C.

10. The dietary supplement composition of claim 1, wherein the active ingredients comprise 885 mg turmeric powder, 100 mg curcumin extract, 15 mg black pepper fruit extract, and 500 mg N-acetylcysteine.

11. The dietary supplement composition of claim 1, wherein the active ingredients comprise 885 mg turmeric powder, 500 mg N-acetylcysteine, and 100 mg Vitamin C.

12. The dietary supplement composition of claim 1, wherein the active ingredients comprise 885 mg turmeric powder and 500 mg N-acetylcysteine.

13. A method of promoting efficient ethanol metabolism, mitigating hangovers from alcohol consumption, enhancing immune function, and/or promoting stress recovery, comprising administering orally a dietary supplement composition to a subject in need thereof, wherein the dietary supplement composition comprises turmeric powder, curcumin extract, black pepper fruit extract, N-acetylcysteine, Vitamin C, or a combination thereof.

14. The method of claim 13, wherein the turmeric powder is in an amount between 200 mg and 2000 mg, the curcumin extract is in an amount between 10 mg and 300 mg, the black pepper fruit extract is in an amount between 5 mg and 50 mg, the N-acetylcysteine is in an amount between 100 mg and 1200 mg, and the Vitamin C is in an amount between 0 mg and 1000 mg.

15. The method of claim 13, wherein the dietary supplement composition comprises 885 mg turmeric powder, 100 mg curcumin extract, 15 mg black pepper fruit extract, 500 mg N-acetylcysteine, and 100 mg Vitamin C.

16. The method of claim 13, wherein the dietary supplement composition comprises 885 mg turmeric powder, 100 mg curcumin extract, 15 mg black pepper fruit extract, and 500 mg N-acetylcysteine.

17. The method of claim 13, wherein the dietary supplement composition comprises 885 mg turmeric powder, 500 mg N-acetylcysteine, and 100 mg Vitamin C.

18. The method of claim 13, wherein the dietary supplement composition comprises 885 mg turmeric powder and 500 mg N-acetylcysteine.

19. The method of claim 13, wherein the black pepper fruit extract provides piperine that enhances the bioavailability of curcumin by at least 10 folds.

20. The method of claim 13, wherein the N-acetylcysteine and Vitamin C act synergistically to enhance antioxidant protection against alcohol-induced oxidative stress and boost immune function.
